(19) United States
(12) Patent Application Publication
(10) Pub. No.: US 2009/0018457 A1
(43) Pub. Date: Jan. 15, 2009

(54) Clip-type monitoring device for wirelessly transmitting the heart rate

(76) Inventor: Chin-Yeh Hung, Taoyuan City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/822,892

(22) Filed: Jul. 11, 2007

(51) Int. Cl. A61B 5/0402

(52) U.S. Cl. 600/509

(57) ABSTRACT

A clip-type monitoring device for wirelessly transmitting a heart rate comprises a housing of which the back is formed with a clip, which may be clipped onto the clothes, and the flank is formed with a jack connected to an external signal; a heart rate monitoring unit formed with multiple electrode pads extending from the housing; a voltage sensor unit provided in the housing so that the voltage variation frequency measured is filtered by the inductors and then transmitted to the filter shaping amplifier circuit to receive an amplified signal; and a radio frequency module having a wireless signal transmission circuit and provided in the housing for transmitting the signal amplified by the voltage sensor unit to a display storage device having a wireless signal receiving circuit. The housing inside which the heart rate monitoring module is provided to accurately measure the pulsation is light and thin and conveniently hanged on the clothes without burden.

### BACKGROUND OF THE INVENTION

1. Field of the Invention

The invention relates to a heart rate monitoring device and particularly to a model that is provided with a heart rate monitoring module and that may be clipped onto the clothes, of which electrode pads are used to monitor the heart rate and a radio frequency module is used to transmit a heart rate signal to a specified storage device to receive and display.

2. Description of the Related Art

Conventionally, people's heart rate remarkably varies before and after people take exercise. During exercise, measuring the variables is very important. To take exercise properly and advantageously, the heart rate must increase to 60% of the maximum rate; however, if the heart rate increases to the maximum rate during exercise, it will do harm to the exerciser. Thus, a user-friendly and precise heart rate measurement device is very important for taking exercise to keep fit.

Next, FIG. 1 shows an electrode belt of heart rate monitor disclosed in U.S. Pat. No. 6,553,247, in which a heart rate monitoring unit 60 is bound on the chest with a belt 61 to monitor the heart rate on and on and wirelessly transmit a signal to a receiver 70 bound around the wrist. However, it is found that the patent is poor with the following conditions when being applied.

1. Generally, the heart rate monitoring unit 60 is tight bound with the belt 61 on the chest to keep close to; however, the lung capacity increases during exercise and thus the belt bound up on the chest causes a user to be uncomfortable and breathe hard.

2. Female chest contour is bigger, so the heart rate monitoring unit 60 cannot stay fully close to the chest, thereby the accuracy of monitoring decreasing.

3. To accurately measure the heart rate, the heart rate monitoring unit 70 cannot be bound outside the clothes but inside the clothes and close to the chest, so the belt 71 is placed certainly after the clothes is taken off, which might be somewhat inconvenient to males but may be implemented none the less; however, it is very inconvenient to females to bind up the belt 71 in a public place; thus, the conventional belt is not properly used by the female.

Consequently, because of the technical defects of described above, the applicant keeps on carving unflaggingly through wholehearted experience and research to develop the present invention, which can effectively improve the defects described above.

### SUMMARY OF THE INVENTION

For this reason, it is a main object of the invention to solve the defects of the existing products and to provide a clip-type monitoring device for wirelessly transmitting a heart rate whoses housing is light and thin and conveniently hanged on the clothes without burden. Meanwhile, the positive and negative pads of the heart rate monitoring unit are conveniently made to stay fully close to the left and right chests. Thus, it is convenient for the female users to take exercise. Besides, the monitoring accuracy can be increased.

It is a further object of the invention to provide a clip-type monitoring device for wirelessly transmitting a heart rate, of which positive and negative electrode pads passes through a human body to be conducted. In consideration of the factors of voltage that is generated when the blood circulates through the human body, that varies with variable heart rate, and that is more distinct regular if the blood in the human body is quite away from the elements, a more accurate heart rate may be obtained.

In order to achieve the above-mentioned objects, the clip-type monitoring device for wirelessly transmitting the heart rate includes:

a) a housing having a clip at the back thereof, the clip being clipped onto the clothes, a jack being formed at the flank thereof for receiving an external signal;

b) a heart rate monitoring unit having a plurality of electrode pads extending out of the housing and staying close to the human body's chest to monitor the pulsation, each of the electrode pads being connected with a conducting wire, a plug being provided at a influx terminal for electrically coupled to the jack such that a measured voltage variation rate is transmittable to the inside of the housing;

c) a voltage sensor unit disposed within the housing and provided with three inductors and a filtering shaping amplifier circuit so that the voltage variation frequency measured by the heart rate monitoring unit is filtered by the inductors and then transmitted to the filtering shaping amplifier circuit to receive an amplified signal;

d) a control interface provided on the surface of housing to control the heart rate monitoring unit and the voltage sensor unit to turn ON and OFF; and

e) a radio frequency module having a wireless signal transmission circuit and provided in the housing for transmitting the signal amplified by the voltage sensor unit to a display storage device provided with a wireless signal receiving circuit.

### BRIEF DESCRIPTION OF THE FIGURES

The accomplishment of this and other objects of the invention will become apparent from the following descriptions and its accompanying figures of which:

FIG. 1 is a schematic view illustrating the structure of a device disclosed in U.S. Pat. No. 6,553,247;

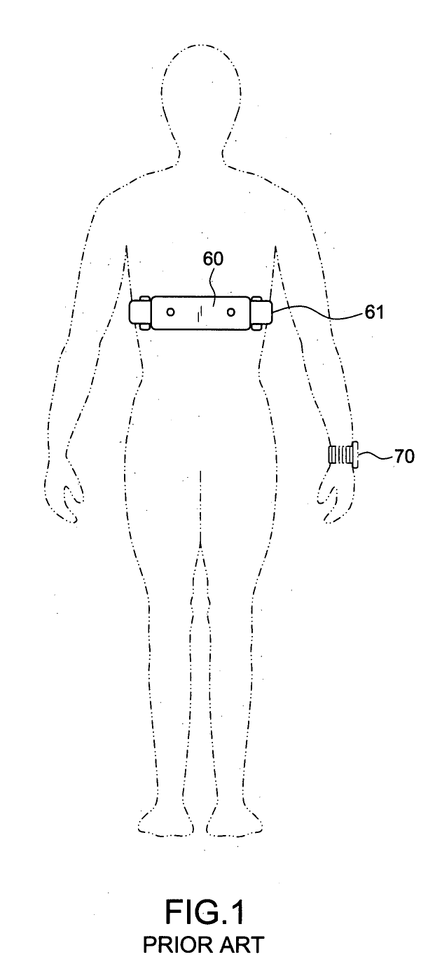

FIG. 2 is a front side view illustrating a preferred embodiment of the invention;

FIGS. 3A and 3B are perspective views of the back of a clip according to the invention;

FIG. 4 is a schematic view illustrating the measurement of heart rate according to the invention;

FIG. 5 is a block diagram of a circuit according to the invention; and

FIG. 6 is a view of an embodiment of the invention.

### DETAILED DESCRIPTION OF THE PREFERRED EMBODIMENT

Now, the present invention will be described more specifically with reference to the following embodiments. It is to be noted that the following descriptions of preferred embodiments of the invention are presented herein for purpose of illustration and description only; it is not intended to be exhaustive or to be limited to the precise form disclosed.

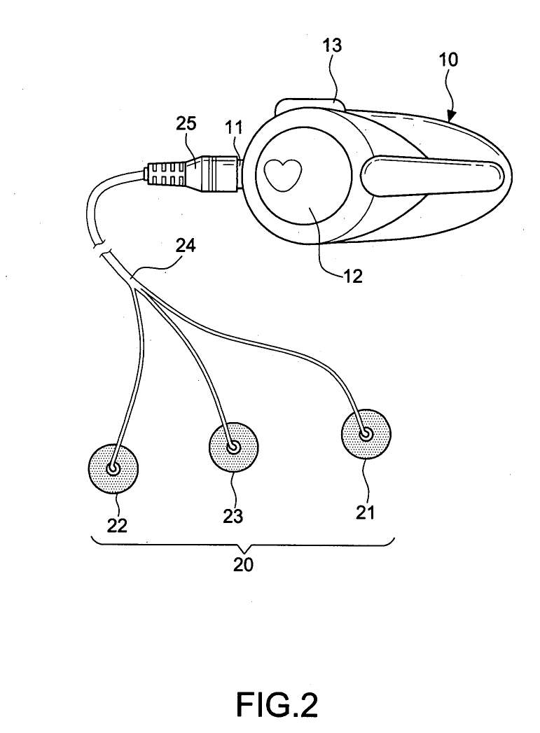

First, with reference to FIGS. 2 through 5, a preferred embodiment of the invention comprises:

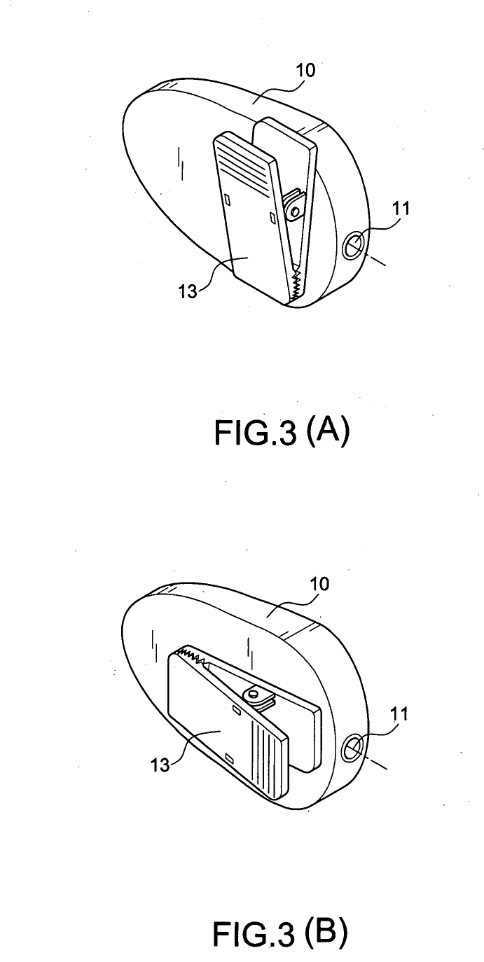

a housing 10 of which the back is formed with a clip 13, as shown in FIGS. 3A and 3B, that may be located vertically or horizontally, and of which the flank is formed with a jack 11 connected to an external signal;

a heart rate monitoring unit 20 being multiple electrode pads extending out of the housing 10, which stay close to the human body's chest to monitor the pulsation, in this embodiment, comprising a positive electrode pad 21, a negative electrode pad 22, and a common electrode pad 23; a lead wire 24 to which each of the electrode pad 21˜23 are provided along a wire influx terminal provided with a plug 25 that may be electrically connected to a jack 11, thereby a measured voltage variation rate being transmitted to the housing 10;

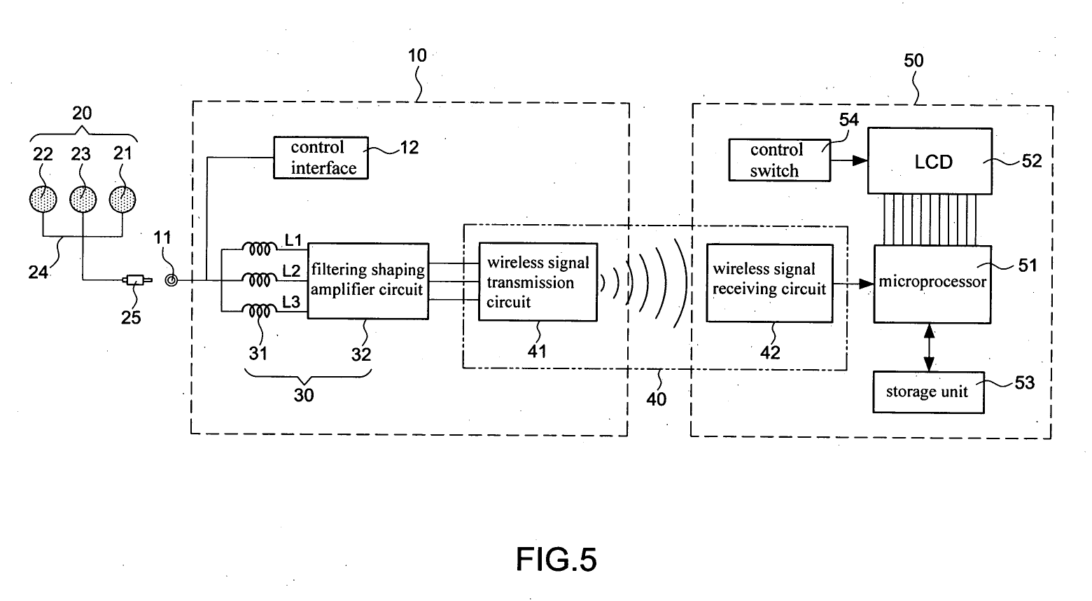

a voltage sensor unit 30, as shown in FIG. 5, which is provided in the housing 10 and provided with 3 inductors 31 and filtering shaping amplifier circuit 32 so that the voltage variation frequency measured by the heart rate monitoring unit 20 is filtered by the inductors 31, L1˜L3, and then transmitted to the filtering shaping amplifier circuit 32 to receive an amplified signal;

a control interface 12 provided on the surface of housing 10 to control the heart rate monitoring unit 20 and the voltage sensor unit 30 to turn ON and OFF; and

a radio frequency module 40 through which a wireless signal transmission circuit 41 provided in the housing 10 transmits the signal amplified by the voltage sensor unit 30 to a display storage device 50 provided with a wireless signal receiving circuit 42.

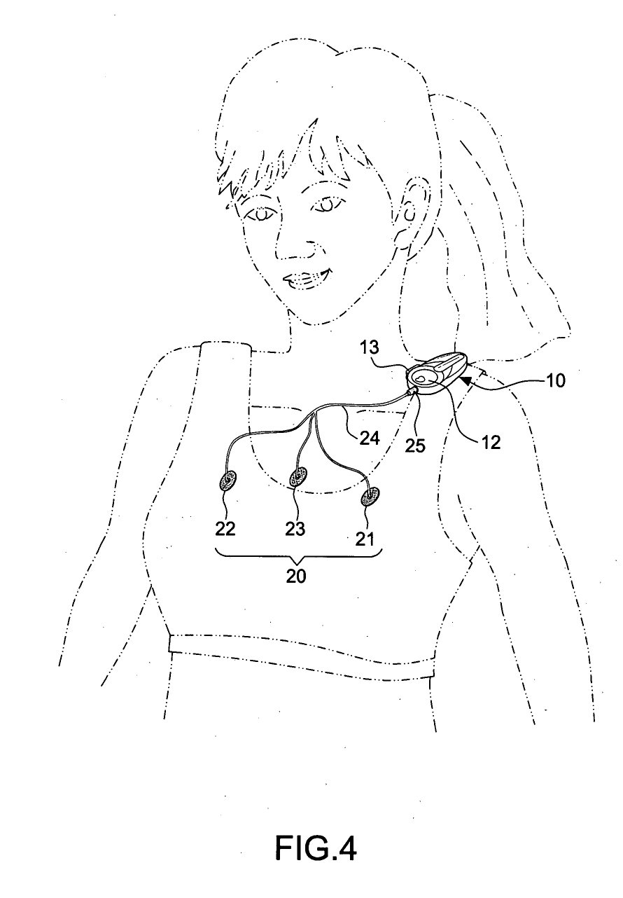

In the invention, to monitor the heart rate, as shown in FIG. 4, the housing body 10 is clipped at a proper site on the clothes and in principle preferably at a site near the chest, and then the three electrode pads 21˜23 serving as the heart rate monitoring unit 20 is stuck to the chest. Each of the 3 electrode pads 21˜23 is formed with a sheet metal or an electrically conductive rubber, both of which are well electrically conductive and keep the pads tight and easily close to the chest. Preferably, the positive and negative electrode pads 21 and 22 are separately stuck to the left and right chest as far as possible; when the blood circulates through the human body, voltage is generated, and the heart rate varies, the frequency of voltage also varies; the more the blood in the human body is away from the elements, the voltage frequency varies remarkably. In the invention, the positive and negative electrode pads 21 and 22 are separately stuck to the left and right chest, so the heart rate may be accurately obtained.

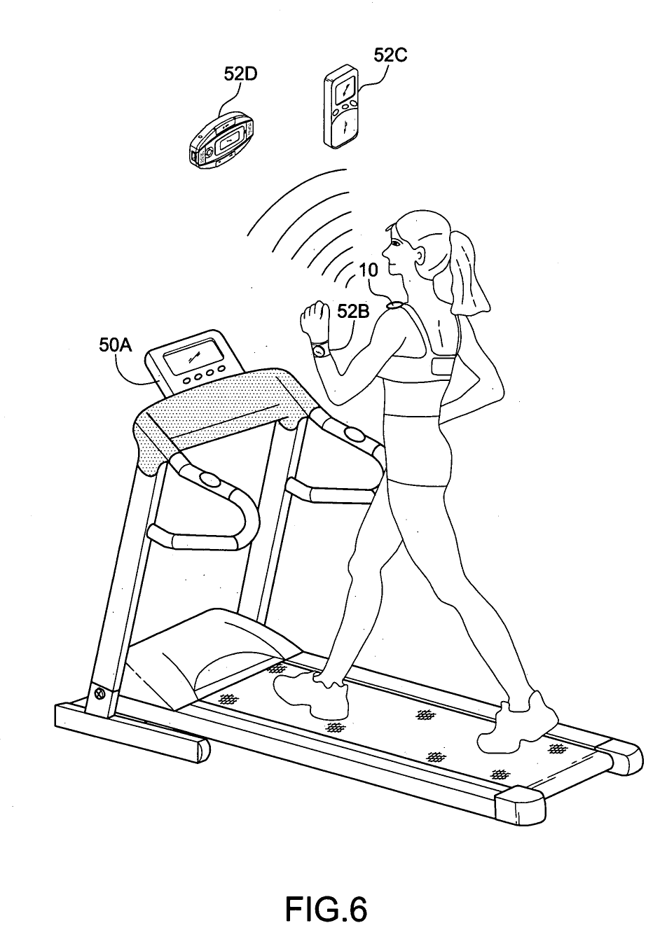

For properly taking exercise, the invention is mainly featured with the light, thin housing body 10 of low volume and weight for easy clipping on the clothes without burden. For this purpose, in the invention, the heart rate monitoring unit 20 is provided with extremely light and thin electrode pads 21˜23 to measure the voltage variation rate of the chest, and the display unit occupying more space further displays it through a peripheral electronic product; namely, the radio frequency module 40 is designed in the invention to transmit a measured heart rate signal through a wireless signal transmission circuit 41 to a display storage device 50 provided with a wireless signal receiving circuit 42. Thanks to amazingly fast technology, most of the current electronic products are provided with a display panel, for example, as shown in FIG. 6, a meter 50 A designed in an exercise machine, a sports wrist watch 50B, a mobile phone 50C, and an MP3/4 player 50D, thereby unnecessary details being not given here. Thus, as long as being provided with the wireless signal receiving circuit 42, the electronic products 50A˜50D may wirelessly transmit signals to the housing body 10.

Further, as shown in FIG. 5, the display storage device 50 provided with the display panel 52 further includes a microprocessor 51, a storage unit 53, and a control switch 54, in which a default program and circuit provided in the microprocessor 51 is used to operate and process the signal inputted by the wireless signal receiving circuit 42 and then to transmit the data to the display panel 52 for a user to read. The storage unit 53 may store the data given by the microprocessor for backup, and the control switch 54 is used to control the display panel 52 to turn ON or OFF for the heart rate data display.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A clip-type monitoring device for wirelessly transmitting the heart rate, comprising:
a) a housing having a clip at the back thereof, the clip being clipped onto the clothes, a jack being formed at the flank thereof for receiving an external signal;
b) a heart rate monitoring unit having a plurality of electrode pads extending out of the housing and staying close to the human body's chest to monitor the pulsation, each of the electrode pads being connected with a conducting wire, a plug being provided at a influx terminal for electrically coupled to the jack such that a measured voltage variation rate is transmittable to the inside of the housing;
c) a voltage sensor unit disposed within the housing and provided with three inductors and a filtering shaping amplifier circuit so that the voltage variation frequency measured by the heart rate monitoring unit is filtered by the inductors and then transmitted to the filtering shaping amplifier circuit to receive an amplified signal;
d) a control interface provided on the surface of housing to control the heart rate monitoring unit and the voltage sensor unit to turn ON and OFF; and
e) a radio frequency module having a wireless signal transmission circuit and provided in the housing for transmitting the signal amplified by the voltage sensor unit to a display storage device provided with a wireless signal receiving circuit.

2. The clip-type monitoring device for wirelessly transmitting the heart rate as recited in claim 1, wherein the electrode pad of the heart rate monitoring unit comprises a positive electrode pad, a negative electrode pad, and a common electrode pad and each of the pads is kept tight close to the surface of the chest.

3. The clip-type monitoring device for wirelessly transmitting the heart rate as recited in claim 1, wherein the display storage device comprises an exercise machine meter provided with the wireless signal receiving circuit and a display panel.

4. The clip-type monitoring device for wirelessly transmitting the heart rate as recited in claim 1, wherein the display storage device comprises a wristwatch provided with the wireless signal receiving circuit and the display panel.

5. The clip-type monitoring device for wirelessly transmitting the heart rate as recited in claim 1, wherein the display storage device comprises a mobile phone provided with the wireless signal receiving circuit and the display panel.

6. The clip-type monitoring device for wirelessly transmitting the heart rate according to claim 1, wherein the display storage device comprises an MP3/4 player provided with the wireless signal receiving circuit and the display panel.

7. The clip-type monitoring device for wirelessly transmitting the heart rate according to claim 1, wherein the display storage device further includes a microprocessor, a storage unit, and a control switch, and wherein a default program and circuit provided in the microprocessor is used to operate and process the signal and then to transmit the data to the display panel, and wherein the storage unit stores the data given by the microprocessor for backup, and wherein the control switch is used to control the display panel to turn ON or OFF for the heart rate data display.
